(19) United States
(12) Patent Application Publication
(10) Pub. No.: US 2025/0082638 A1
(43) Pub. Date: Mar. 13, 2025

(54) Dosing Regime For Treatment of Chronic Hand Eczema

(71) Applicant: Pfizer Inc., New York, NY (US)

(72) Inventors: Gary Lap-Chiu Chan, West Warwick, RI (US); Maureen Patricia Neary, Princeton, NJ (US); Hernan Valdez, Girona (ES)

(73) Assignee: Pfizer Inc., New York, NY (US)

(21) Appl. No.: 18/359,250

(22) PCT Filed: Jan. 31, 2022
(86) PCT No.: PCT/IB2022/050818

§ 371 Date: Jul. 26, 2023

Related U.S. Application Data
(60) Provisional application No. 63/144,912, filed on Feb. 2, 2021.

(51) Int. Cl. A61K 31/519; A61K 45/06

(52) U.S. Cl. CPC A61K 31/519; A61K 45/06

(57) ABSTRACT

Method for treating moderate to severe chronic hand eczema in a subject in need thereof, which method comprises the step of administering to said subject a therapeutically effective amount of the compound of N-((1S,3S)-3-(methyl(7H-pyrrolo[2,3-d]pyrimidin-4-yl)amino)cyclobutyl)propane-1-sulfonamide, or a pharmaceutically acceptable salt thereof.

### FIELD OF THE INVENTION

The present invention provides methods for treating moderate to severe chronic hand eczema using compounds and analogues which inhibit certain kinases including Janus Kinase (JAK) in particular inhibitors of JAK1, and more particularly, the compound N-((1S,3S)-3-(methyl(7H-pyrrolo[2,3-d]pyrimidin-4-yl)amino)cyclobutyl)propane-1-sulfonamide, or a pharmaceutically acceptable salt thereof.

### BACKGROUND OF THE INVENTION

Hand eczema (HE), also referred to as hand dermatitis, is a common dermatological condition characterized by erythema, hyperkeratosis, vesiculation, scaling, dryness, papules, fissures, itching, and pain, and most frequently affects the back of the hands and fingers. It is the most common occupational skin disease, as the skin on the hands often comes into contact with various irritants and contact allergens resulting in damage. HE is a heterogenous condition with various etiologies (ie, endogenous factors, such as atopic dermatitis and exogenous factors, such as exposure to allergens and irritants) and morphologies and can range from very mild to severe and from acute to chronic. Meding et al. found that the extent of eczema involvement at a patient's first examination, a history of childhood eczema, and an onset of HE before the age of 20, were all associated with poor long-term prognosis. Meding, et al., J Invest Dermatol 2002; 118 (4): 719-723; Meding, et al., J Invest Dermatol 2005; 124 (5): 893-897. Patients with all three of these risk factors had almost twice the chance of still having HE after 15 years, compared with patients who had none of the risk factors (72% versus 35%, respectively).

Chronic hand eczema (CHE) is defined by the European Society of Contact Dermatitis (ESCD) as HE that persists for greater than 3 months or recurs at least twice within a 12-month period despite adequate therapy and patient adherence. However, it has sometimes been defined as HE that lasts longer than 6 months. Acute HE should be treated quickly to prevent the development of CHE. The longer HE persists, the more likely it is to become a chronic condition even if the initial causative agent is avoided. CHE can result not only in anxiety, social phobia, and low self-esteem due to its visibility on the hand and poor understanding within the general population, it can also significantly impact the quality of life and economic outcomes of patients, contributing to morbidity and lost earnings. The long-term prognosis for severe CHE is poor.

Irrespective of the etiology, CHE involves multiple inflammatory pathways Th1, Th17, Th22, Th2. In a literature review of HE worldwide (most results came from Western countries), the point prevalence (weighted average) of HE was 3.0%, the 1-year prevalence (weighted average) was 9.1% to 9.7%, the lifetime prevalence (weighted average) was 14.0%, and was found to affect women more often than men (1-year prevalence of 10.5% vs. 6.4% and lifetime prevalence of 18.4% vs. 10.0%, respectively). Prevalence of CHE in subjects less than 19 years old is low. Among certain occupational groups involved in wet work or exposed to irritants and/or allergic substances, such as hairdressers, cleaners, cooks, and healthcare workers, the prevalence rates account for up to 30% of HE.

Prior treatment methods have been suboptimal. Topical treatments include bland emollients, corticosteroid creams and ointments, topical immunomodulators, topical retinoids, and coal tar and derivatives, while physical therapies include irradiation with ultraviolet (UV) light. Because calcineurin inhibitors such as tacrolimus and pimecrolimus may reduce the need for corticosteroids but may not be useful in achieving remission, calcineurin inhibitors and corticosteroids could be combined. Systemic treatments include azathioprine, methotrexate, cyclosporine, alitretinoin and other retinoids, and oral corticosteroids in short courses. Alitretinoin, an endogenous retinoid, is the first systemic treatment approved in the European Union (but not in the US) to treat severe CHE that does not respond to potent topical corticosteroids.

CHE can result in anxiety, low self-esteem, and social phobia, and is associated with a decrease in quality of life. CHE can also be a major cause of lost earnings because symptoms can limit patients' abilities to do their jobs. Available pharmaceutical therapies are limited, do not always completely abate the inflammatory process, and may have significant adverse effects. Disclosed herein is the discovery that compounds and analogues which inhibit certain kinases such as JAK1 and particularly the compound N-((1S,3S)-3-(methyl(7H-pyrrolo[2,3-d]pyrimidin-4-yl)amino)cyclobutyl)propane-1-sulfonamide are useful for treating. Accordingly, described herein are methods of reducing the severity of CHE symptoms in a human subject, with more rapid onset of action and lower incidence of adverse effects.

### SUMMARY OF THE INVENTION

The present invention provides a method for treating moderate to severe chronic hand eczema in a subject in need thereof, which method comprises the step of administering to said subject a therapeutically effective amount of certain JAK inhibitors, in particular, JAK1 inhibitors.

According to a first aspect of the invention there is provided a method for treating moderate to severe chronic hand eczema in a subject in need thereof, which method comprises the step of administering to said subject a therapeutically effective amount of N-((1S,3S)-3-(methyl(7H-pyrrolo[2,3-d]pyrimidin-4-yl)amino)cyclobutyl)propane-1-sulfonamide, or a pharmaceutically acceptable salt thereof. Described below are a number of embodiments (E) of this first aspect of the invention, where for convenience E1 is identical thereto.

E1. A method for treating moderate to severe chronic hand eczema in a subject in need thereof, which method comprises the step of administering to said subject a therapeutically effective amount N-((1S,3S)-3-(methyl(7H-pyrrolo[2,3-d]pyrimidin-4-yl)amino)cyclobutyl)propane-1-sulfonamide, or a pharmaceutically acceptable salt thereof.

E2. The method of E1, wherein the therapeutically effective amount is selected from the group consisting of 50, 100 or 200 mg.

E3. The method of E1, wherein the therapeutically effective amount is 50 mg.

E4. The method of E1, wherein the therapeutically effective amount is 100 mg.

E5. The method of E1, wherein the therapeutically effective amount is 200 mg.

E6. The method of any E1 to E5, wherein the therapeutically effective amount is administered QD.

E7. The method of any of E1 to E6, wherein the N-((1S,3S)-3-(methyl(7H-pyrrolo[2,3-d]pyrimidin-4-yl)amino)cyclobutyl)propane-1-sulfonamide, or a pharmaceutically acceptable salt thereof is administered to said subject for up to 16 weeks.

E8. The method of any of E1 to E7, wherein the N-((1S,3S)-3-(methyl(7H-pyrrolo[2,3-d]pyrimidin-4-yl)amino)cyclobutyl)propane-1-sulfonamide, or a pharmaceutically acceptable salt thereof is administered to said subject for up to 52 weeks.

E9. The method of any of E1 to E8, wherein whereby said subject achieves an Investigator Global Assessment (IGA) response score of 0-1 and ≥2 points reduction in score from baseline.

E10. The method of E9, wherein said subject maintains said IGA response.

E11. The method of any of E1 to E9, whereby said subject achieves an IGA response score of 0-1 and ≥2 points reduction in score from baseline by Week 16.

E12. The method of any of E1 to E11, whereby said subject achieves an improvement in Total Lesion Severity Score (TLSS) of at least about 50 to about 70% reduction from baseline TLSS by Week 16.

E13. The method of any of E1 to E12, whereby said subject achieves a response of at least a 4-point improvement on Worst Itch Numerical Rating Scale (Worst Itch NRS4) by Week 16.

E14. The method of any of E1 to E13, wherein said subject maintains said improvement in the Worst Itch ENRS4 score.

E15. The method of any of E1 to E14, whereby said subject achieves at least a 20 percent improvement in the Work Productivity and Activity Impairment Questionnaire (WPAI-CHE) Scale by Week 16.

E16. The method of any of E15, wherein said subject maintains the improvement in the WPAI-CHE score.

E17. The method of any of E1 to E16, whereby flare as measured by IGA score is maintained at less than or equal to 3.

E18. The method of any of E1 to E17, wherein the improvement in response is within 14 days.

E19. The method of any of E1 to E18, further comprising co-administering a topical agent selected from the group consisting of a crisaborole, corticosteroid, a calcineurin inhibitor, pimecrolimus and tacrolimus.

E20. The method of E19, wherein the topical co-administration is by means of an ointment or cream.

E21. Use of N-((1S,3S)-3-(methyl(7H-pyrrolo[2,3-d]pyrimidin-4-yl)amino)cyclobutyl)propane-1-sulfonamide, or a pharmaceutically acceptable salt thereof, for the preparation of a medicament for the treatment of chronic hand eczema according to any of E1 to E20.

Accordingly, the invention provides a method of treating moderate to severe chronic hand eczema in a subject comprising:
orally administering to the subject for up to 16 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent;
whereby the subject achieves an Investigator Global Assessment (IGA) response score of 0-1 and ≥2 points reduction in score from baseline by Week 16. In certain other embodiments, the invention provides the method, further comprising orally administering to the subject for up to 52 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent, wherein the subject maintains said IGA response.

The invention further provides a method of treating moderate to severe chronic hand eczema in a subject comprising:
orally administering to the subject for up to 52 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent;
whereby the subject achieves an IGA response score of 0-1 and ≥2 points reduction in score from baseline by Week 16.

The invention also provides a method of treating moderate to severe chronic hand eczema in a subject comprising:
orally administering to the subject for up to 16 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50,100 or 200 mg QD of N-((1S,3S)-3-(methyl(7H-pyrrolo[2,3-d]pyrimidin-4-yl)amino)cyclobutyl)propane-1-sulfonamide free base equivalent;
whereby the subject achieves an improvement in Total Lesion Severity Score (TLSS) of at least about 50 to about 70% reduction from baseline TLSS by Week 16. In certain other embodiments, the invention provides the method, further comprising orally administering to the subject for up to 52 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50 mg or 100 mg QD of N-((1S,3S)-3-(methyl(7H-pyrrolo[2,3-d]pyrimidin-4-yl)amino)cyclobutyl)propane-1-sulfonamide free base equivalent, wherein the subject maintains said improvement in Total Lesion Severity Score.

The invention additionally provides a method of treating moderate to severe chronic hand eczema in a subject comprising:
orally administering to the subject for up to 16 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent;
whereby the subject achieves a response of at least a 4-point improvement on Worst Itch Numerical Rating Scale (Worst Itch NRS4) by Week 16. In other embodiments, the invention provides the method, further comprising orally administering to the subject for up to 52 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent, wherein the subject maintains said improvement in the Worst Itch NRS4 score.

The invention also provides a method of treating moderate to severe chronic hand eczema in a subject comprising:
orally administering to the subject for up to 16 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent;
whereby the subject achieves a 4-point improvement in the Worst Pain Numerical Rating Scale (Worst Pain NRS4) by Week 16. In other embodiments, the invention provides the method, further comprising orally administering to the subject for up to 52 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent, wherein the subject maintains said improvement in the Worst Pain NRS4 score.

The invention further provides a method of treating moderate to severe chronic hand eczema in a subject comprising:
orally administering to the subject for up to 16 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent;
whereby the subject achieves a 20 percent improvement in the Work Productivity and Activity Impairment Questionnaire (WPAI-CHE) Scale by Week 16.

The invention additionally provides the method, further comprising orally administering to the subject for up to 52 weeks N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50, 100 or 200 mg QD of N-((1S,3S)-3-(methyl(7H-pyrrolo[2,3-d]pyrimidin-4-yl)amino)cyclobutyl)propane-1-sulfonamide free base equivalent, wherein the subject maintains said improvement in the WPAI-CHE score.

The invention further provides any of the above methods, wherein said improvement in response is within 14 days.

The invention also provides any of the above methods, further comprising co-administering a topical agent selected from the group consisting of a crisaborole, corticosteroid, a calcineurin inhibitor, pimecrolimus and tacrolimus.

The invention additionally provides the above methods, wherein the amount of N-((1S,3S)-3-(methyl(7H-pyrrolo[2,3-d]pyrimidin-4-yl)amino)cyclobutyl)propane-1-sulfonamide free base equivalent is 100 mg.

The invention further provides the above methods, wherein the amount of N-((1S,3S)-3-(methyl(7H-pyrrolo[2,3-d]pyrimidin-4-yl)amino)cyclobutyl)propane-1-sulfonamide free base equivalent is 200 mg.

The invention also provides any of the above methods, further comprising orally administering to the subject N-((1S,3S)-3-(methyl(7H-pyrrolo[2,3-d]pyrimidin-4-yl)amino)cyclobutyl)propane-1-sulfonamide, or a pharmaceutically acceptable salt thereof, in an amount sufficient to deliver 50 mg, 100 or 200 mg QD of N-((1S,3S)-3-(methyl(7H-pyrrolo[2,3-d]pyrimidin-4-yl)amino)cyclobutyl)propane-1-sulfonamide free base equivalent, whereby flare as measured by IGA score is maintained at less than or equal to 3.

The present invention is further understood from the following description given by way of example only. While the present invention is not so limited, an appreciation of various aspects of the invention are gained through the following discussion and the examples.

As used herein, “subject” refers to humans.

The term “adverse effect” (AE) is any untoward medical occurrence in a subject or clinical study participant, temporally associated with the administration of the JAK inhibitor.

The term “QD” or “Q.D.” means one administered dose per day.

The term “treating” or “treatment” means an alleviation of symptoms associated with a disease, disorder or condition, or halt of further progression or worsening of those symptoms. Depending on the disease and condition of the subject, the term “treatment” as used herein may include one or more of curative, palliative and prophylactic treatment. Treatment can also include administering a pharmaceutical formulation of the present invention in combination with other therapies.

The term “therapeutically-effective” indicates the capability of an agent to prevent, or improve the severity of the disorder, while avoiding adverse side effects typically associated with alternative therapies. The phrase “therapeutically-effective” is to be understood to be equivalent to the phrase “effective for the treatment, prevention, or amelioration”, and both are intended to qualify the amount of each agent for use in the combination therapy which will achieve the goal of improvement in the severity of disease, or pain or other symptom thereof, and the frequency of incidence over treatment of each agent by itself, while avoiding adverse side effects typically associated with alternative therapies.

The term “moderate-to-severe” is used in reference to subjects suffering from chronic hand eczema who are assessed using the Investigator Global Assessment (IGA) tool to have an IGA score of 3 or 4 at baseline.

“Pharmaceutically acceptable” means suitable for use in a “subject.”

### DETAILED DESCRIPTION OF THE INVENTION

The present invention relates to the above methods which comprise, inter alia, the step of administering to said subject a therapeutically effective amount of a pharmaceutical composition comprising certain JAK inhibitors, especially JAK1 inhibitors, and more particularly, N-((1S,3S)-3-(methyl(7H-pyrrolo[2,3-d]pyrimidin-4-yl)amino)cyclobutyl)propane-1-sulfonamide, or a pharmaceutically acceptable salt thereof.

In therapeutic use for treating moderate to severe chronic hand eczema in a subject, a compound of the present invention or its pharmaceutical compositions can be administered orally, parenterally, topically, rectally, transmucosally, or intestinally. Parenteral administrations include indirect injections to generate a systemic effect or direct injections to the afflicted area. Topical administrations include the treatment of skin or organs readily accessible by local application, for example, eyes or ears. It also includes transdermal delivery to generate a systemic effect. The rectal administration includes the form of suppositories. The preferred routes of administration are oral, topical and parenteral.

Pharmaceutical compositions of the present invention may be manufactured by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant invention. Such excipients and carriers are described, for example, in Remington's Pharmaceutical Sciences, Mack Pub. Co., New Jersey (1991). The formulations of the invention can be designed to be short-acting, fast-releasing, long-acting, and sustained-releasing. Thus, the pharmaceutical formulations can also be formulated for controlled release or for slow release.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

The compounds of the invention may be prepared by any method known in the art. In particular, the compounds of the invention can be prepared by the procedures described by reference to the prior art references in which they are disclosed.

For those compounds which inhibit JAK1 specifically, including N-((1S,3S)-3-(methyl(7H-pyrrolo[2,3-d]pyrimidin-4-yl)amino)cyclobutyl)propane-1-sulfonamide, preparative methods are disclosed in U.S. Pat. No. 9,035,074, the contents of which are incorporated herein in their entirety.

### EXAMPLES

The following non-limiting examples are presented merely to illustrate the present invention. The skilled person will understand that there are numerous equivalents and variations not exemplified but which still form part of the present teachings.

### Example 1

### Methods of Treatment of Chronic Hand Eczema

Subjects take the medication set forth herein orally once daily, and typically swallow the tablets whole with approximately one cup of ambient temperature water, without any manipulation or chewing. Tablets may be taken with or without food.

### Example 2

### Investigator Global Assessment (IGA)

The subject is examined at predetermined intervals after receiving the medication in accordance with the Investigator Global Assessment scale for measuring the global/overall severity of skin lesions of the hands and feet.

### Investigator Global Assessment Measure for Chronic Hand Eczema

| | | | | Induration/ | | |
|---|---|---|---|---|---|---|
| | | Scaling | Lichenification^{b}/ | Papulation^{d}/ | | |
| IGA Grade | Erythema^{a} | (Desquamation) | Hyperkeratosis^{c} | Edema | Vesicles^{e} | Fissures^{f} |
| 0 = Clear | Absent^{a} | Absent | Absent | Absent | Absent | Absent |
| 1 = Almost | Slight | A few fine | Faint | Barely | A few | 1-2 small |
| Clear | erythema | scales | lichenification/ | perceptible | vesicles, | fissures |
| | (trace pink) | | hyperkeratosis | | without | |
| | | | | | oozing/ | |
| | | | | | crusting | |
| 2 = Mild | Definite | Fine | Mild | Perceptible/ | Vesicles | Small |
| | erythema | scales | lichenification/ | palpable | over a | fissures |
| | (faint pink) | over a | hyperkeratosis | | limited area, | over a |
| | | limited | | | without | limited area |
| | | area | | | oozing/ | |
| | | | | | crusting | |
| 3 = Moderate | Clearly | Scaling | Clearly visible | Clearly | Vesicles | Small |
| | perceptible | over | lichenification and/ | perceptible/ | over multiple | fissures |
| | erythema | multiple | or | palpable, over | areas, with | over |
| | (pink-red) | areas | hyperkeratosis | multiple areas | or without | multiple |
| | | | | | oozing/ | areas or 1-2 |
| | | | | | crusting | deep |
| | | | | | | fissures |
| 4 = Severe | Marked | Widespread | Marked, thick or | Marked or | Widespread | Widespread |
| | erythema | scaling | widespread | widespread | vesicles, | small |
| | (deep or | | lichenification and/ | induration, | bullae (ie, | fissures or >2 |
| | bright red) | | or | papules or | coalescing | deep |
| | | | hyperkeratosis | edema | vesicles) or | fissures |
| | | | | | oozing/ | |
| | | | | | crusting | |

^{a}Physiological palmar erythema should not be included in the evaluation. Erythema can present differently in various Fitzpatric skin types, e.g., redness in lighter skin types and violaceous and/or dusky hues in darker skin types.
^{b}Mostly involving the dorsal aspect of the hand, presenting as accentuation of skin markings, due to rubbing and scratching.
^{c}Mostly involving the palm due to corneocyte hyperproliferation.
^{d}Mostly involving the dorsal aspect of the hand
^{e}Oozing/crusting can result from ruptured vesicles.
^{f}The number of fissures in the descriptor text refers to the findings on each hand affected by chronic hand eczema

This may scale be applied to the hand(s) affected by eczema (both hands if both hands are affected), including both dorsal and palmar aspects. For each feature (erythema, scaling, lichenification/hyperkeratosis, induration/papulation/edema, vesicles, fissures), the severity descriptor that best fits the patient's global assessment of the hand(s) may be assigned. The overall IGA grade is assigned based on the totality of the severity of the individual features. To assign IGA 0 ‘clear’ or 1 ‘almost clear’, the subject may a) NOT have any individual features with grade 3 or grade 4 severity, and b) NOT have ≥3 individual features with grade 2 or higher severity. This scale may be used to evaluate chronic eczema of the feet, when feet are also involved. Hands and feet are evaluated separately.

### Example 3

### Body Surface Area (BSA)

The number of hand units of skin afflicted with chronic hand eczema in a body region can be used to determine the extent (%) to which a body region is involved with chronic hand eczema. When measuring, the hand unit refers to the size of each individual subject's palm plus the volar surface of all the digits in a closed position.

### Example 4

### Total Lesion Severity Score

The subject is examined at predetermined intervals after receiving the medication in accordance with the Total Lesion Severity Score (TLSS), a scale that measures the severity of skin lesions of the hands.

| Parameter | Description of Severity* |
|---|---|
| Erythema ^{a},^{b} | 0 = Absent; 1 = Faint erythema; |
| | 2 = Prominent redness; 3 = Deep intense |
| | red color |
| Scaling ^{a},^{b} | 0 = Absent; 1 = Slight flaking over |
| | limited areas, mostly fine scales; |
| | 2 = Flaking over widespread area(s), |
| | coarser scales; 3 = Desquamation |
| | covering over 30% of the hand, |
| | with coarse thick scales |
| Hyperkeratosis/ | 0 = Absent; 1 = Mild thickening with |
| Lichenification ^{a},^{b} | exaggerated skin lines over limited |
| | areas; 2 = Palpable thickening over |
| | widespread area(s); 3 = Prominent |
| | thickening over widespread area(s) |
| | with exaggeration of normal skin |
| | markings |
| Vesiculation^{a},^{b} | 0 = Absent; 1 = Scattered vesicles |
| | affecting up to 10% of hand, without |
| | erosion; 2 = Scattered or clustered |
| | vesicles affecting up to 30% of hand, |
| | without visible erosion or excoriation; |
| | 3 = High density of vesicles |
| | extending over large area(s), or |
| | with erosion or excoriation |
| Edema ^{a},^{b} | 0 = Absent; 1 = Dermal swelling over |
| | less than 10% of hands; 2 = Definite |
| | dermal swelling over more than 10% |
| | of hand; 3 = Dermal swelling with |
| | skin induration over widespread area(s) |
| Fissures without | 0 = Absent; 1 = Cracked skin |
| pain ^{b} | affecting a small area of the hand; |
| | 2 = Cracked skin affecting multiple |
| | areas of the hand; 3 = One or more |
| | deep fissures and causing bleeding |
| Fissures ^{a} | 0 = Absent; 1 = Cracked skin affecting |
| | a small area of the hand; |
| | 2 = Cracked skin affecting multiple |
| | areas of the hand and causing pain; |
| | 3 = One or more deep fissures and |
| | causing bleeding or severe pain |
| Pruritus or Pain ^{a} | 0 = Absent; 1 = Occasional slight |
| | discomfort a few times a day; |
| | 2 = Intermittent causing discomfort |
| | frequently during the day; |
| | 3 = Persistent or interfering with sleep |

^{a} 7 parameters used to assess TLSS (0-21).
^{b} 6 parameters used for TLSS (0-18).
*0 = absent; 1 = mild; 2 = moderate; 3 = severe.

### Example 5

### Hand Eczema Severity Index (HECSI)

The subject is examined at predetermined intervals after receiving the medication in accordance with the Hand Eczema Severity Index (HECSI), a scale that measures the severity and extent of skin lesions of the hands.

Hand Eczema Severity Index

| | | | | | |
|---|---|---|---|---|---|
| | | Fingers | Palms | Back | |
| | | (except | of | of | |
| Clinical Signs | Fingertips | fingertips) | Hands | Hands | Wrists |

| | |
|---|---|
| Erythema (E) | 4-Point Intensity Scale = 0-3 |
| Induration/ | (0, no skin changes; 1, mild; 2, |
| Papulation (I) | moderate; 3, severe) |
| Vesicles (V) | |
| Fissuring (F) | |
| Scaling (S) | |
| Oedema/Edema (O) | |
| SUM of | Maximum Intensity Scale for each of 5 |
| Clinical Signs = | Location = 6 (Clinical Signs) * 3 |
| E + 1 + V + | (Intensity Scale) = 18 |
| F + S + O | |
| Extent | 5-Point Extent Scale = 0-4 for each of 5 |
| | Locations (Affected area of each location |
| | to be described as % and |
| | programmatically converted to 5-point scale) |
| Total HECSI | Maximum HECSI Score = 18 |
| Score = | (Clinical Sign*Intensity Scale) |
| SUM * Extent | * 4 (Extent) * 5 (Locations) = 360 |

### Example 6

### Worst Pain NR Numerical Rating Scale (NRS)

The subject is examined at predetermined intervals after receiving the medication in accordance with the Worst Pain NRS, a numerical rating scale for pain of the hands with a 24-hour recall period.

### Example 7

### Worst Itch Numerical Rating Scale (NRS)

The subject is examined at predetermined intervals after receiving the medication in accordance with the Worst Itch NRS, a numerical rating scale for itch of the hands with a 24-hour recall period.

### Example 8

### Work Productivity and Activity Impairment Questionnaire (WPAI-CHE)

CHE has been shown to impact work productivity, including both manual and office-based work (Grant L, et al., Adv Ther, 2020, 37:692-706). This may involve the need to take more breaks while at work to apply topical treatments and may also result in work absences. The Work Productivity and Activity Impairment Questionnaire: Chronic Hand Eczema (WPAI-CHE) was used to assess work burdens. The WPAI has demonstrated validity, reliability and sufficient predictive value to measure the impact of disease on absenteeism, presenteeism, and overall productivity in a manner that can also be monetized (Reilly M C, et al., PharmacoEconomics, 1993; 4 (5): 353-365).

1. A method for treating moderate to severe chronic hand eczema in a subject in need thereof, which method comprises the step of administering to said subject a therapeutically effective amount N-((1S,3S)-3-(methyl(7H-pyrrolo[2,3-d]pyrimidin-4-yl)amino)cyclobutyl)propane-1-sulfonamide, or a pharmaceutically acceptable salt thereof.

2. The method of claim 2, wherein the therapeutically effective amount is selected from the group consisting of 50, 100 or 200 mg.

3. The method of claim 2, wherein the therapeutically effective amount is 50 mg.

4. The method of claim 2, wherein the therapeutically effective amount is 100 mg.

5. The method of claim 2, wherein the therapeutically effective amount is 200 mg.

6. The method of 1, wherein the therapeutically effective amount is administered QD.

7. The method of claim 1, wherein the N-((1S,3S)-3-(methyl(7H-pyrrolo[2,3-d]pyrimidin-4-yl)amino)cyclobutyl)propane-1-sulfonamide, or a pharmaceutically acceptable salt thereof is administered to said subject for up to 16 weeks.

8. The method of claim 1, wherein the N-((1S,3S)-3-(methyl(7H-pyrrolo[2,3-d]pyrimidin-4-yl)amino)cyclobutyl)propane-1-sulfonamide, or a pharmaceutically acceptable salt thereof is administered to said subject for up to 52 weeks.

9. The method of claim 1, wherein whereby said subject achieves an Investigator Global Assessment (IGA) response score of 0-1 and ≥2 points reduction in score from baseline.

10. The method of claim 9, wherein said subject maintains said IGA response.

11. The method of claim 1, whereby said subject achieves an IGA response score of 0-1 and ≥2 points reduction in score from baseline by Week 16.

12. The method of claim 1, whereby said subject achieves an improvement in Total Lesion Severity Score (TLSS) of at least about 50 to about 70% reduction from baseline TLSS by Week 16.

13. The method of claim 1, whereby said subject achieves a response of at least a 4-point improvement on Worst Itch Numerical Rating Scale (Worst Itch NRS4) by Week 16.

14. The method of claim 1, wherein said subject maintains said improvement in the Worst Itch NRS4 score.

15. The method of claim 1, whereby said subject achieves at least a 20 percent improvement in the Work Productivity and Activity Impairment Questionnaire (WPAI-CHE) Scale by Week 16.

16. The method of claim 15, wherein said subject maintains the improvement in the WPAI-CHE score.

17. The method of claim 1, whereby flare as measured by IGA score is maintained at less than or equal to 3.

18. The method of claim 1, wherein the improvement in response is within 14 days.

19. The method of claim 1, further comprising co-administering a topical agent selected from the group consisting of a crisaborole, corticosteroid, a calcineurin inhibitor, pimecrolimus and tacrolimus.

20. The method of claim 19, wherein the topical co-administration is by means of an ointment or cream.

21. (canceled)
